(19) United States
(12) Patent Application Publication
(10) Pub. No.: US 2025/0264427 A1
(43) Pub. Date: Aug. 21, 2025

(54) GAS SENSOR

(71) Applicant: TDK Corporation, Tokyo (JP)

(72) Inventors: Kei TANABE, Tokyo (JP); Yoshikatsu TANAKA, Tokyo (JP)

(21) Appl. No.: 19/197,096

(22) Filed: May 2, 2025

Related U.S. Application Data
(63) Continuation of application No. PCT/JP2022/043031, filed on Nov. 21, 2022.

(51) Int. Cl. G01N 27/14; G01N 33/00

(52) U.S. Cl. CPC G01N 27/14; G01N 33/0027

(57) ABSTRACT

Disclosed herein is a gas sensor that includes: first and second thermistors; first and second heaters configured to heat the first and second thermistors, respectively; and a control circuit. The control circuit is configured to, in a gas measurement operation, generate an output signal based on a detection signal appearing at a connection point between the first and second thermistors by heating the first and second heaters. The control circuit is configured to, in a resistance measurement operation, measure a resistance value of the first thermistor with a series connection of the first and second thermistors disconnected. The control circuit is configured to correct a value of the output signal based on the resistance value of the first thermistor measured in the resistance measurement operation.

### BACKGROUND OF THE ART

### Field of the Art

The present disclosure relates to a gas sensor.

### Description of Related Art

International Publication WO 2020/031517 discloses a gas sensor configured to calculate the concentration of a gas to be detected based on the level of a detection signal appearing at the connection point of two series-connected thermistors. The gas sensor described in International Publication WO 2020/031517 heats a thermistor constituting a detection element and a thermistor constituting a reference element to 150° C. and 300° C., respectively, to acquire a detection signal and then heats the thermistor as the detection element and thermistor as the reference element to 300° C. and 150° C., respectively, to eliminate a difference in thermal history between the two thermistors.

However, even in the gas sensor described in Patent Document 1, the temperature characteristics of the thermistor may change with age.

### SUMMARY

A gas sensor according to the present disclosure includes: a detection circuit including a first thermistor and a second thermistor; a first heater configured to heat the first thermistor; a second heater configured to heat the second thermistor; and a control circuit configured to control a connection relation in the detection circuit and the first and second heaters. The control circuit is configured to, in a gas measurement operation, generate an output signal indicating a concentration of a gas to be measured based on a detection signal appearing at a connection point between the first and second thermistors by connecting the first and second thermistors in series and heating the first and second heaters. The control circuit is configured to, in a resistance measurement operation, measure a resistance value of the first thermistor with a series connection of the first and second thermistors disconnected. The control circuit is configured to correct a value of the output signal based on the resistance value of the first thermistor measured in the resistance measurement operation.

### BRIEF DESCRIPTION OF THE DRAWINGS

The above features and advantages of the present disclosure will be more apparent from the following description of some embodiments taken in conjunction with the accompanying drawings, in which:

FIG. 1 is a circuit diagram illustrating the configuration of a gas sensor 1 according to a first embodiment of the technology described herein;

FIGS. 2A and 2B are circuit diagrams of the resistance measuring circuit 11;

FIG. 3 is a timing chart for explaining the operation of the gas sensor 1;

FIG. 4 is a graph characteristics of the thermistors Rd1 and Rd2;

illustrating the temperature

FIG. 5 is a graph illustrating the relation between the temperature of the thermistors Rd1, Rd2 and their sensitivity to CO₂ gas;

FIG. 6 is a circuit diagram illustrating the configuration of a gas sensor 2 according to a second embodiment of the technology disclosed herein;

FIG. 7 is a timing chart for explaining the operation of the gas sensor 2;

FIG. 8 is a circuit diagram illustrating the configuration of a gas sensor 3 according to a third embodiment of the technology disclosed herein; and

FIG. 9 is a circuit diagram illustrating the configuration of a gas sensor 4 according to a fourth embodiment of the technology disclosed herein.

### DETAILED DESCRIPTION OF THE EMBODIMENTS

The present disclosure describes a gas sensor in which a measurement error due to aging of the thermistor is reduced.

Some embodiments of the present disclosure will be explained below in detail with reference to the accompanying drawings.

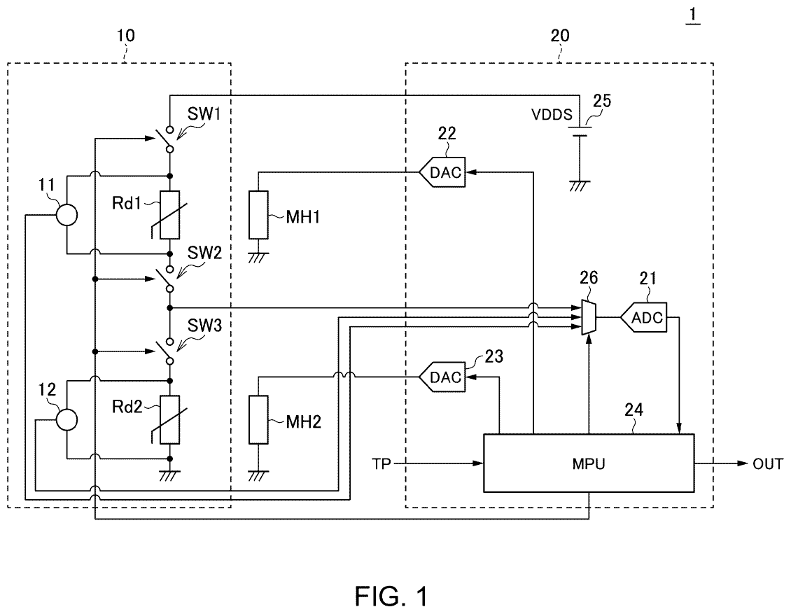

FIG. 1 is a circuit diagram illustrating the configuration of a gas sensor 1 according to a first embodiment of the technology described herein.

As illustrated in FIG. 1, the gas sensor 1 according to the first embodiment includes: a detection circuit 10 including thermistors Rd1, Rd2 and resistance measurement circuits 11, 12; heaters MH1 and MH2 for heating the thermistors Rd1 and Rd2, respectively; and a control circuit for controlling the detection circuit 10 and heaters MH1 and MH2. Although not particularly limited, the gas sensor) 1 according to the present embodiment is a thermal conduction type gas sensor for detecting the concentration of CO₂ gas in the atmosphere.

The detection circuit 10 includes the thermistors Rd1 and Rd2, the resistance measurement circuits 11 and 12, and switches SW1 to SW3. The thermistors Rd1 and Rd2 are detection elements made of a material having a negative temperature coefficient of resistance, such as a composite metal oxide, amorphous silicon, polysilicon, or germanium. Both the thermistors Rd1 and Rd2 detect the concentration of CO₂ gas, but have different operating temperatures, as described below. Here, the thermistor Rd1 constitutes a detection element, and the thermistor Rd2 constitutes a reference element.

As illustrated in FIG. 1, the switch SW1 is connected between a power supply 25 for supplying a power supply potential VDDS and the thermistor Rd1. The switches SW2 and SW3 are connected between the thermistors Rd1 and Rd2. Thus, when the switches SW1 to SW3 are turned ON, the thermistors Rd1 and Rd2 are connected in series between the power supply and the ground. A potential appearing between the switches SW2 and SW3 in this state, i.e., a detection signal appearing at the connection point between the thermistors Rd1 and Rd2 is supplied to the control circuit 20. On the other hand, when the switches SW1 to SW3 are turned OFF, the series connection of the thermistors Rd1 and Rd2 is released, and the thermistors Rd1 and Rd2 are separated from each other. The resistance measurement circuits 11 and 12 measure the resistance values of the thermistors Rd1 and Rd2, respectively, in a state where the switches SW1 to SW3 are tuned OFF.

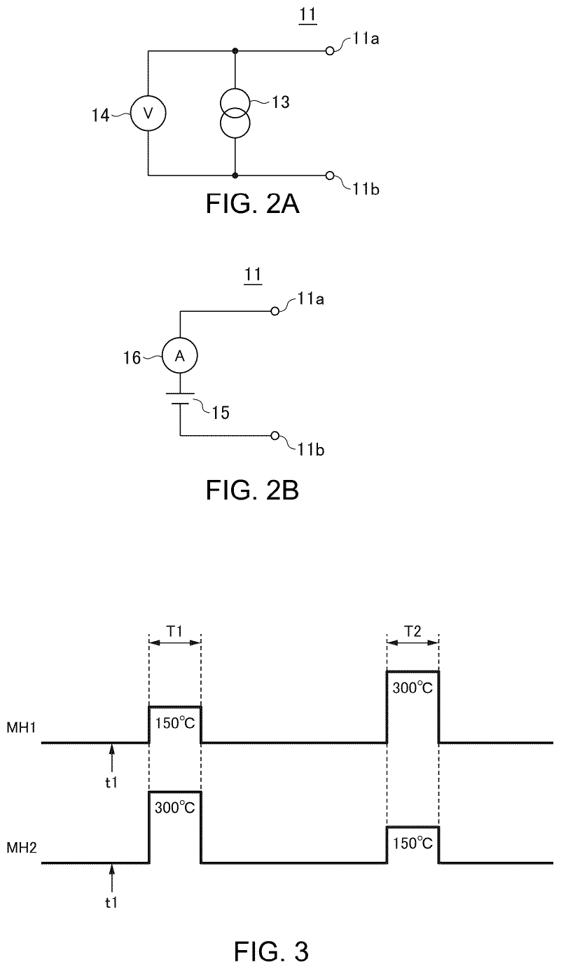

As illustrated in FIG. 2A, the resistance measurement circuit 11 may have a configuration in which a constant current source 13 and a voltmeter 14 are connected in parallel between one end 11a and the other end 11b. With this configuration, when a constant current is made to flow from the constant current source 13 to the thermistor Rd1 in a state where the thermistor Rd1 is connected between the one end 11a and the other end 11b, a voltage to be generated between the one end 11a and the other end 11b is determined by the resistance value of the thermistor Rd1. This voltage is measured with the voltmeter 14 and supplied to the control circuit 20. This allows the control circuit 20 to acquire the directly-measured resistance value of the thermistor Rd1.

Alternatively, as illustrated in FIG. 2B, the resistance measurement circuit 11 may have a configuration in which a constant voltage source 15 and an ammeter 16 are connected in series between the one end 11a and the other end 11b. With this configuration, when a predetermined voltage is applied from the constant voltage source 15 to the thermistor Rd1 in a state where the thermistor Rd1 is connected between the one end 11a and the other end 11b, a current flowing between the one end 11a and the other end 11b is determined by the resistance value of the thermistor Rd1. This current is measured with the ammeter 16 and supplied to the control circuit 20. This allows the control circuit 20 to acquire the directly-measured resistance value of the thermistor Rd1.

The same applies to the configuration of the resistance measurement circuit 12, and the control circuit 20 can acquire the directly-measured resistance value of the thermistor Rd2.

The control circuit 20 includes an AD converter (ADC) 21, DA converters (DAC) 22 and 23, an MPU 24, the power supply 25, and a multiplexer 26. The multiplexer 26 supplies, under the control of the MPU 24, either a detection signal appearing at the connection point between the thermistors Rd1 and Rd2 or resistance measurement signals output respectively from the resistance measurement circuits 11 and 12 to the AD converter 21. A plurality of the AD converters 21 may be used in place of the multiplexer 26.

The MPU 24 controls the switches SW1 to SW3 to control the connection relation in the detection circuit 10. The MPU 24 turns OFF the switches SW1 to SW3 during resistance measurement operation. The AD converter 21 sequentially AD-converts the resistance measurement signals supplied in this state from the resistance measurement circuits 11 and 12 and supplies the resultant signals to the MPU 24. The MPU 24 calculates the resistance values of the thermistors Rd1 and Rd2 based on the AD-converted resistance measurement signals.

On the other hand, during gas measurement operation, the MPU 24 turns ON the switches SW1 to SW3. The AD converter 21 AD-converts a detection signal appearing in this state at the connection point between the thermistors Rd1 and Rd2 and supplies the resultant signal to the MPU 24. The MPU 24 calculates an output signal OUT indicating the concentration of CO₂ gas based on the AD-converted detection signal. In the calculation of the output signal OUT, the value of the output signal OUT is corrected based on the resistance values of the thermistors Rd1 and Rd2 calculated during the resistance measurement operation.

The DA converters 22 and 23 DA-convert the digital value supplied from the MPU 24 to apply predetermined voltages to the heater resistors MH1 and MH2, respectively. In other words, the heating temperatures of the heater resistors MH1 and MH2 are controlled by the MPU 24.

The following describes the operation of the gas sensor 1 according to the present embodiment.

FIG. 3 is a timing chart for explaining the operation of the gas sensor 1 according to the present embodiment.

As illustrated in FIG. 3, the gas sensor 1 according to the present embodiment performs the gas measurement operation in period T1 and dummy heating operation in period T2. The gas measurement operation and dummy heating operation are performed alternately. Further, the gas sensor 1 performs the resistance measurement operation at time t1, which is the timing immediately before period T1 in which the gas measurement operation is performed. During the resistance measurement operation, heating of the thermistors Rd1 and Rd2 by the heater resistors MH1 and MH2 is stopped. Therefore, the resistance measurement operation performed at time t1 is executed with thermistors Rd1 and Rd2 under the environmental temperature.

In the present embodiment, a temperature signal TP indicating the current environmental temperature is supplied to the MPU 24. This allows the MPU 24 to acquire the current environmental temperature and the resistance values of the thermistors Rd1 and Rd2 under the current environmental temperature and thereby to calculate, based on the thus acquired information, the resistance values of the thermistors Rd1 and Rd2 at a predetermined reference temperature (e.g., 25° C.). The MPU 24 stores therein design resistance values of the thermistors Rd1 and Rd2 at a predetermined reference temperature (e.g., 25° C.). The MPU 24 compares the design resistance values of the thermistors Rd1 and Rd2 and actually acquired resistance values thereof to correct the value of the output signal based on the resistance values of the thermistors.

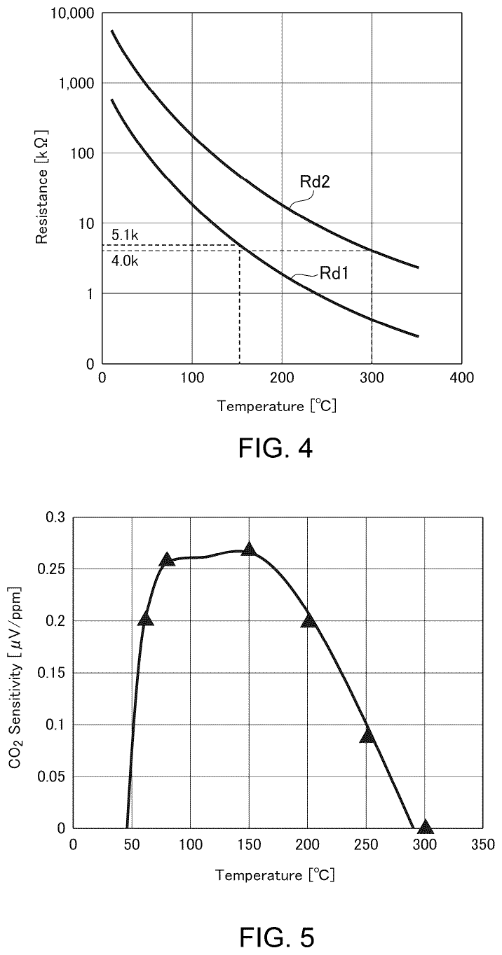

In the gas measurement operation performed during period T1, the heater resistor MH1 is heated to 150° C. and the heater resistor MH2 is heated to 300° C. under the control of the MPU 24. As illustrated in FIG. 4, the temperature characteristics of thermistors Rd1 and Rd2 are different from each other, and they are designed such that the resistance value of the thermistor Rd1 heated to 150° C. and the resistance value of the thermistor Rd2 heated to 300° C. are close to each other. In the example illustrated in FIG. 4, the resistance value of the thermistor Rd1 heated to 150° C. is 5.1 kΩ, and the resistance value of the thermistor Rd2 heated to 300° C. is 4.0 kΩ. The resistance value of the thermistor Rd1 heated to 150° C. and the resistance value of the thermistor Rd2 heated to 300° C. may be approximately the same.

FIG. 5 is a graph illustrating the relation between the temperature of the thermistors Rd1, Rd2 and their sensitivity to CO₂ gas. As can be seen from the graph of FIG. 5, the sensitivity of the thermistors Rd1 and Rd2 to CO₂ gas varies significantly depending on the temperature, and the sensitivity of the thermistors Rd1, Rd2 to CO₂ gas is almost zero in the temperature range below 40° C. or above 300° C. The sensitivity of the thermistors Rd1, Rd2 to CO₂ gas is maximum at about 150° C.

When CO₂ gas is present in the measurement atmosphere with the thermistor Rd1 as the detection element heated to 150° C., the heat dissipation characteristics of the thermistor Rd1 change according to the concentration. Such a change appears as a change in the resistance value of the thermistor Rd1. On the other hand, even when CO₂ gas is present in the measurement atmosphere with the thermistor Rd2 as the reference element heated to 300° C., the heat dissipation characteristics of the thermistor Rd2 hardly change according to the concentration. Therefore, the change in the resistance value of the thermistor Rd2 heated to 300° C. due to the concentration of CO₂ gas is sufficiently smaller than the change in the resistance value of the thermistor Rd1 heated to 150° C. due to the concentration of CO₂ gas. There is no problem if the resistance value of the thermistor Rd2 heated to 300° C. due to the concentration of CO₂ gas hardly changes.

As a result, the level of the detection signal appearing at the connection point between the thermistors Rd1 and RD2 changes according to the concentration of CO₂ gas in the measurement atmosphere. The detection signal is supplied to the MPU 24 through the AD converter 21, and the MPU 24 generates an output signal OUT indicating the concentration of CO₂ gas based on the supplied detection signal. In calculating the output signal OUT, a correction is made based on the result of the resistance measurement operation performed at time t1.

As described above, the resistance measurement operation performed at time t1 is performed under the environmental temperature at which the sensitivity of the thermistors Rd1 and Rd2 to CO₂ gas is almost zero, so that the resistance values of the thermistors Rd1 and Rd2 can be accurately measured regardless of the concentration of CO₂ gas in the environment during the resistance measurement operation. Therefore, it is possible to calculate the resistance value of the thermistor Rd1 heated to 150° C. and the resistance value of the thermistor Rd2 heated to 300° C. when the concentration of CO₂ gas is the same as the concentration of CO₂ gas in the atmosphere under normal conditions (about 400 ppm). As a result, the output signal OUT indicating the concentration of CO₂ gas can be calculated using the detection signal that actually appears at the connection point between the thermistors Rd1 and Rd2, the resistance value under normal conditions of the thermistor Rd1 heated to 150° C., and the resistance value under normal conditions of the thermistor Rd2 heated to 300° C.

In the dummy heating operation performed in period T2, the heater resistor MH1 is heated to 300° C., and the heater resistor MH2 is heated to 150° C. under the control of the MPU 24. This eliminates the thermal history difference between the thermistors Rd1 and Rd2 during the gas measurement operation performed in period T1.

In this way, the gas sensor 1 according to the present embodiment actually measures the resistance values of the thermistors Rd1 and Rd2 using the resistance measurement circuits 11 and 12, so that even if the thermistors Rd1 and Rd2 have changed with age, the value of the output signal OUT can be properly corrected. Moreover, the measurement of the resistance values of the thermistors Rd1 and Rd2 using the resistance measurement circuits 11 and 12 is performed with heating by the heater resistors MH1 and MH2 stopped, so there is no measurement error due to the concentration of CO₂ gas present in the atmosphere. In addition, by performing the resistance measurement operation immediately before the period T1, the influence of residual heat is eliminated, and it is possible to measure the resistance values of the thermistors Rd1 and Rd2 under a more accurate environmental temperature.

However, if the actual environmental temperature is significantly deviated from a predetermined reference temperature (e.g., 25° C.), the resistance measurement operation may be affected by CO₂ gas. Therefore, the MPU 24 may disable the resistance measurement operation when the environmental temperature is outside the predetermined temperature range, e.g., when it exceeds 40° C. In this case, the resistance measurement operation itself may be skipped, or the calculation operation after the resistance measurement operation may be skipped. Alternatively, although the resistance measurement operation and the calculation operation are performed, the values obtained thereby may be ignored.

In addition, in the present embodiment, the gas measurement operation is performed in period T1, and the dummy heating operation is performed in period T2, so that the thermistors Rd1 and Rd2 undergo approximately the same aging change. Taking this into consideration, it is not essential to actually measure the resistance values of both thermistors Rd1 and Rd2, and it is also possible to actually measure the resistance value of only one of the thermistors Rd1 and Rd2 and calculate the resistance value of the other thermistor Rd2 based on that result. For example, by measuring the resistance value of the thermistor Rd1 at room temperature, both the resistance value of the thermistor Rd1 heated to 150° C. and the resistance value of the thermistor Rd2 heated to 300° C. may be calculated.

In the present embodiment, heating by the heater resistors MH1 and MH2 is stopped during the resistance measurement operation, but heating by the heater resistors MH1 and MH2 may be performed as long as the sensitivity of the thermistors Rd1 and Rd2 to CO₂ gas is sufficiently low. As an example, the thermistors Rd1 and Rd2 may be heated to 300° C. by the heater resistors MH1 and MH2 during the resistance measurement operation.

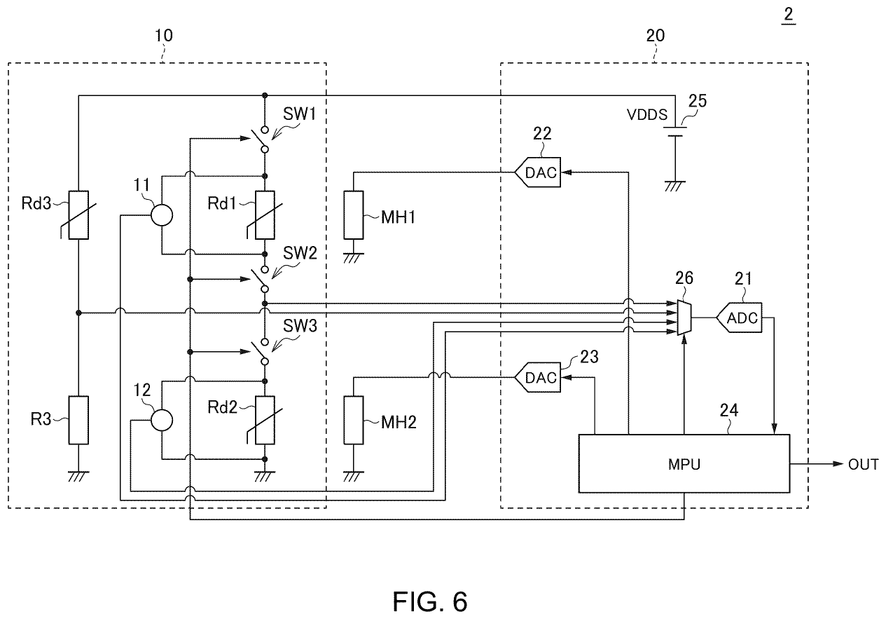

FIG. 6 is a circuit diagram illustrating the configuration of a gas sensor 2 according to a second embodiment of the technology disclosed herein.

As illustrated in FIG. 6, the gas sensor 2 according to the second embodiment differs from the gas sensor 1 according to the first embodiment in that the detection circuit 10 includes a thermistor Rd3 and a fixed resistor R3. Other basic configurations are the same as those of the gas sensor 1 according to the first embodiment, so the same reference numerals are given to the same elements, and overlapping description will be omitted.

The thermistor Rd3 and fixed resistor R3 are connected in series between the power supply 25 and the ground, and a temperature signal TP appears the connection point therebetween. The temperature signal TP is supplied to the AD converter 21 through the multiplexer 26. The AD converter 21 AD-converts the temperature signal TP and supplies the resultant signal to the MPU 24.

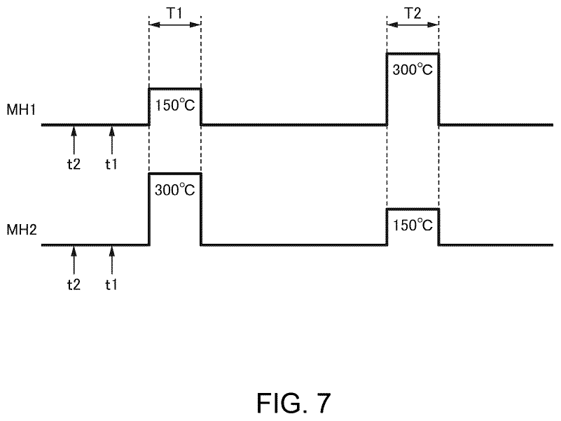

FIG. 7 is a timing chart for explaining the operation of the gas sensor 2 according to the present embodiment.

As illustrated in FIG. 7, the gas sensor 2 according to the present embodiment acquires the temperature signal TP at time t2, which is the timing immediately before period T1, and performs the resistance measurement operation at time t1. The order of time t1 and time t2 does not matter, but it is preferable that the time difference between the two is small. This makes it possible to more accurately measure the environmental temperature during the resistance measurement operation.

As exemplified in the second embodiment, the detection circuit 10 itself may generate the temperature signal TP.

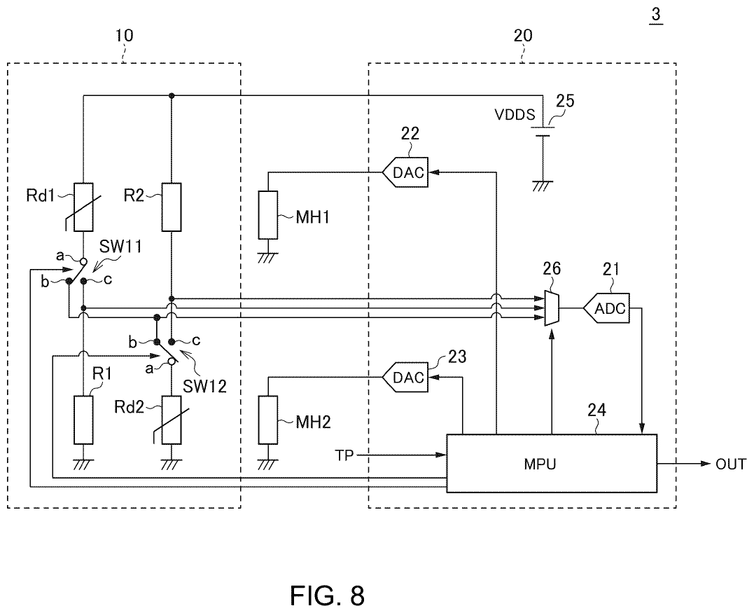

FIG. 8 is a circuit diagram illustrating the configuration of a gas sensor 3 according to a third embodiment of the technology disclosed herein.

As illustrated in FIG. 8, the gas sensor 3 according to the third embodiment differs from the gas sensor 1 according to the first embodiment in that the detection circuit 10 includes fixed resistors R1 and R2 and switches SW11 and SW12. Other basic configurations are the same as those of the gas sensor 1 according to the first embodiment, so the same reference numerals are given to the same elements, and overlapping description will be omitted.

The switches SW11 and SW12 each have one common node a and two selection nodes b and C, and one of the selection nodes b and c is connected to the common node a. For both of the switches SW1 and SW2, the selection node b is selected during the gas measurement operation and dummy heating operation, and the selection node c is selected during resistance measurement operation.

As illustrated in FIG. 8, the common node a of the switch SW11 is connected to one end of thermistor Rd1, the selection node b of the switch SW11 is connected to the selection node b of the switch SW12, and the selection node c of the switch SW11 is connected to one end of the fixed resistor R1. The other end of the thermistor Rd1 is connected to the power supply 25 that supplies a power supply potential VDDS, and the other end of the fixed resistor R1 is connected to a wiring to which a ground potential GND is supplied. The common node a of the switch SW12 is connected to one end of thermistor Rd2, the selection node b of the switch SW12 is connected to the selection node b of the switch SW11, and the selection node c of the switch SW12 is connected to one end of the fixed resistor R2. The other end of the fixed resistor R2 is connected to the power supply 25 that supplies a power supply potential VDDS, and the other end of the thermistor Rd2 is connected to a wiring to which a ground potential GND is supplied.

As a result, during the gas measurement operation and dummy heating operation in which the selection node b is selected, the thermistors Rd1 and Rd2 are connected in series between the power supply 25 and the ground. On the other hand, during the resistance measurement operation in which the selection node c is selected, the thermistor Rd1 and fixed resistor R1 are connected in series between the power supply 25 and the ground, and the fixed resistor R2 and thermistor Rd2 are connected in series between the power supply 25 and the ground. As a result, during the resistance measurement operation, the resistance value of the thermistor Rd1 can be calculated based on the potential that appears at the connection point between the thermistor Rd1 and the fixed resistor R1, and the resistance value of the thermistor Rd2 can be calculated based on the potential that appears at the connection point between the fixed resistor R2 and the thermistor Rd2.

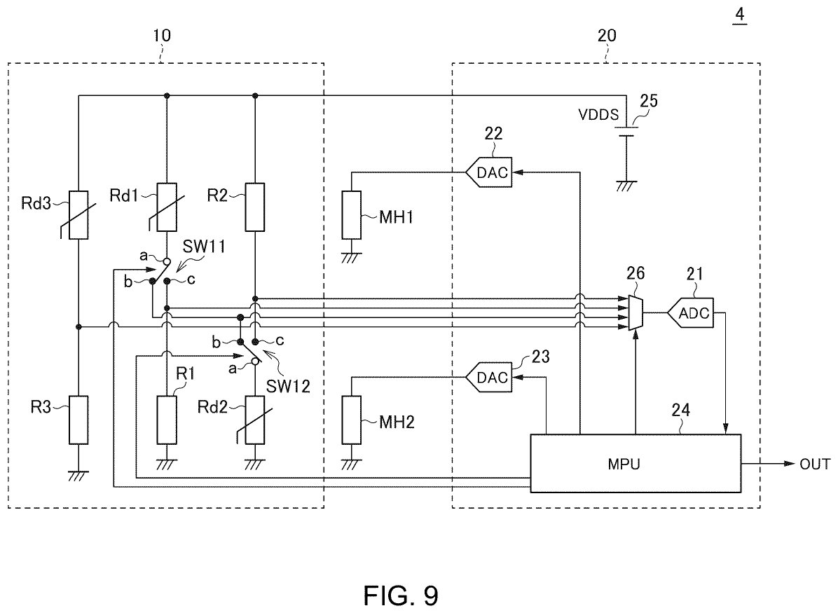

FIG. 9 is a circuit diagram illustrating the configuration of a gas sensor 4 according to a fourth embodiment of the technology disclosed herein.

As illustrated in FIG. 9, the gas sensor 4 according to the fourth embodiment differs from the gas sensor 3 according to the third embodiment in that the detection circuit 10 includes a thermistor Rd3 and a fixed resistor R3. Other basic configurations are the same as those of the gas sensor 3 according to the third embodiment, so the same reference numerals are given to the same elements, and overlapping description will be omitted.

The thermistor Rd3 and fixed resistor R3 are connected in series between the power supply 25 and the ground, and the temperature signal TP appears at the connection point therebetween. The temperature signal TP is supplied to the AD converter 21 through the multiplexer 26. The AD converter 21 AD-converts the temperature signal TP and supplies the resultant signal to the MPU 24.

The operation of the gas sensor 4 according to the present embodiment is as illustrated in FIG. 7, where the temperature signal TP is acquired at time t2, which is the timing immediately before the period T1, and the resistance measurement operation is performed at time t1.

While some embodiments of the technology according to the present disclosure have been described, the technology according to the present disclosure is not limited to the above embodiments, and various modifications may be made within the scope of the present disclosure, and all such modifications are included in the technology according to the present disclosure.

For example, although the measurement target gas is CO₂ gas in the above embodiments, the present invention is not limited to this. Further, the sensor part used in the present invention need not necessarily be a thermal conduction type sensor, but may be a sensor of other types such as a catalytic combustion type. As an example, when the measurement target gas is CO gas, a catalytic combustion type sensor part can be used.

The technology according to the present disclosure includes the following configuration examples, but not limited thereto.

A gas sensor according to the present disclosure includes: a detection circuit including a first thermistor and a second thermistor; a first heater configured to heat the first thermistor; a second heater configured to heat the second thermistor; and a control circuit configured to control a connection relation in the detection circuit and the first and second heaters. The control circuit is configured to, in a gas measurement operation, generate an output signal indicating a concentration of a gas to be measured based on a detection signal appearing at a connection point between the first and second thermistors by connecting the first and second thermistors in series and heating the first and second heaters. The control circuit is configured to, in a resistance measurement operation, measure a resistance value of the first thermistor with a series connection of the first and second thermistors disconnected. The control circuit is configured to correct a value of the output signal based on the resistance value of the first thermistor measured in the resistance measurement operation. Since the resistance value of the first thermistor is thus actually measured, it is possible to correct a measurement error caused by aging of the first and second thermistors.

In the above gas sensor, the control circuit may be configured to stop heating the first heater during the resistance measurement operation. This makes it possible to measure the resistance value of the first thermistor under the environmental temperature.

In the above gas sensor, the control circuit may be configured to calculate a resistance value of the first thermistor at a predetermined temperature based on the resistance value of the first thermistor measured in the resistance measurement operation and an environmental temperature. This makes it possible to more accurately calculate the resistance value of the first thermistor at a predetermined temperature.

In the above gas sensor, the control circuit may be configured to disable a measurement of the resistance value of the first thermistor when the environmental temperature is outside a predetermined temperature range. This makes it possible to avoid correcting the output signal based on the resistance value of the first thermistor calculated under an environment with a large measurement error.

In the above gas sensor, the control circuit may be configured to further measure a resistance value of the second thermistor with disconnecting the series connection of the first and second thermistors during the resistance measurement operation and may be configured to correct the value of the output signal based on the resistance values of the first and second thermistors. Since the resistance values of the first and second thermistors are thus actually measured, it becomes possible to more accurately correct a measurement error caused by aging of the first and second thermistors.

In the above gas sensor, the control circuit may be configured to stop heating the second heater during the resistance measurement operation. This makes it possible to measure the resistance value of the second thermistor under the environment temperature.

In the above gas sensor, the detection circuit may further include a switch connected between first thermistor and the second thermistor, and the control circuit may be configured to turn ON the switch during the gas measurement operation and turn OFF the switch during the resistance measurement operation. This makes it possible to actually measure the resistance value of the first thermistor using a resistance measurement circuit.

In the above gas sensor, the detection circuit may further include a first fixed resistor, and the control circuit may be configured to measure the resistance value of the first thermistor based on a potential appearing at a connection point between the first thermistor and the first fixed resistor by connecting the first thermistor and the first fixed resistor in series during the resistance measurement operation. This makes it possible to actually measure the resistance value of the first thermistor without using the resistance measurement circuit.

In the above gas sensor, the detection circuit may further include a second fixed resistor, and the control circuit may be configured to measure a resistance value of the second thermistor based on a potential appearing at a connection point between the second thermistor and the second fixed resistor by connecting the second thermistor and the second fixed resistor in series during the resistance measurement operation. This makes it possible to actually measure the resistance value of the second thermistor without using the resistance measurement circuit.

What is claimed is:

1. A gas sensor comprising:
a detection circuit including a first thermistor and a second thermistor;
a first heater configured to heat the first thermistor;
a second heater configured to heat the second thermistor; and
a control circuit configured to control a connection relation in the detection circuit and the first and second heaters,
wherein the control circuit is configured to:
in a gas measurement operation, generate an output signal indicating a concentration of a gas to be measured based on a detection signal appearing at a connection point between the first and second thermistors by connecting the first and second thermistors in series and heating the first and second heaters;
in a resistance measurement operation, measure a resistance value of the first thermistor with a series connection of the first and second thermistors disconnected; and
correct a value of the output signal based on the resistance value of the first thermistor measured in the resistance measurement operation.

2. The gas sensor as claimed in claim 1, wherein the control circuit is configured to stop heating the first heater during the resistance measurement operation.

3. The gas sensor as claimed in claim 2, wherein the control circuit is configured to calculate a resistance value of the first thermistor at a predetermined temperature based on the resistance value of the first thermistor measured in the resistance measurement operation and an environmental temperature.

4. The gas sensor as claimed in claim 3, wherein the control circuit is configured to disable a measurement of the resistance value of the first thermistor when the environmental temperature is outside a predetermined temperature range.

5. The gas sensor as claimed in claim 1,
wherein the control circuit is configured to further measure a resistance value of the second thermistor with disconnecting the series connection of the first and second thermistors in the resistance measurement operation; and
wherein the control circuit is configured to correct the value of the output signal based on the resistance values of the first and second thermistors.

6. The gas sensor as claimed in claim 5, wherein the control circuit is configured to stop heating the second heater during the resistance measurement operation.

7. The gas sensor as claimed in claim 1,
wherein the detection circuit further includes a switch connected between the first thermistor and the second thermistor, and
wherein the control circuit is configured to turn ON the switch during the gas measurement operation and turn OFF the switch during the resistance measurement operation.

8. The gas sensor as claimed in claim 1,
wherein the detection circuit further includes a first fixed resistor, and
wherein the control circuit is configured to measure the resistance value of the first thermistor based on a potential appearing at a connection point between the first thermistor and the first fixed resistor by connecting the first thermistor and the first fixed resistor in series during the resistance measurement operation.

9. The gas sensor as claimed in claim 8,
wherein the detection circuit further includes a second fixed resistor, and
wherein the control circuit is configured to measure a resistance value of the second thermistor based on a potential appearing at a connection point between the second thermistor and the second fixed resistor by connecting the second thermistor and the second fixed resistor in series during the resistance measurement operation.
